(19) United States
(12) Patent Application Publication
(10) Pub. No.: US 2010/0114154 A1
(43) Pub. Date: May 6, 2010

(54) SURGICAL BONE CLAMP

(76) Inventor: Christopher Snell, Alexis, IL (US)

Correspondence Address: MICHAEL BEST & FRIEDRICH LLP, Two Prudential Plaza, 180 North Stetson Avenue, Suite 2000, CHICAGO, IL, 60601, US

(21) Appl. No.: 12/263,086

(22) Filed: Oct. 31, 2008

(51) Int. Cl. A61B 17/00; A61B 17/04

(52) U.S. Cl. 606/205; 606/324

(57) ABSTRACT

The surgical bone clamp securely grasps and removes bone and other tissue during surgery, such as total knee replacement surgery. In one embodiment, the device includes a handle portion, a clamp portion, and a ratchet portion. The handle portion includes two handle arms that are pivotally connected, and the ratcheting portion permits the handle arms to be spaced at any one of a plurality of step settings. One of the handle arms provides a finger loop to engage the thumb, while the other handle arm provides a finger loop to engage at least one opposing finger. The clamp portion includes two serrated clamping jaws designed to engage the bone or other tissue surface and to provide adequate gripping force to allow for removal of the bone piece or tissue from the body cavity. The device provides significant mechanical advantage to the user, may be operated with one hand, and may include removable clamping jaws.

### FIELD OF THE INVENTION

This invention generally pertains to a clamping device used in orthopedic surgery, with a particular embodiment of the invention being described, without limitation, in the context of total knee arthroplasty. More particularly, with respect to knee surgery, the invention relates to a bone clamp used to resect a proximal portion of the tibia.

### BACKGROUND OF THE INVENTION

Total knee arthroplasty involves the replacement of a proximal portion of the tibia, a distal portion of the femur, and the posterior portion of the patella with artificial components. Specifically, an important part of total knee replacement involves resection of a proximal portion of the tibia. As used herein, the term “proximal” means closer to the heart and “distal” means further from the heart.

During total knee replacement, the head of the tibia is cut away and resected, leaving the end of the tibia as a planar surface to complement an artificial knee component. The planar surface lends itself to adhesion of the implant. The portion of the proximal tibia that is cut away and removed generally measures approximately 5.0 to 7.5 cm wide, medial to lateral, and from 0.4 to 1.0 cm long, proximal to distal.

Medical professionals may use any number of various clamps and forceps to remove the proximal portion of the tibia during knee replacement. Often, the clamps used to remove the bone are designed for specific tasks other than tibial resection. As such, a surgeon must be very careful when removing tibial bone segments in total knee replacement, such as removing the proximal head of the tibia from the body cavity, as lack of a proper tool combined with improper use of another clamp may cause bone breakage or fragmentation.

Moreover, osteoarthritis and degenerative bone conditions are common in patients undergoing total knee replacement. Patients who suffer from such conditions have bones that are in a weakened condition even before surgery begins. Weakened bones are easier to break, making use of improperly designed clamps risky to the patient during knee arthroplasty. If a clamp breaks the tibial piece during surgery, the bone must then be removed in multiple pieces, increasing both the total length of surgery and the risk of complications to the patient. Some clamps may even cause fragmentation of weakened bones, and it is difficult and time-consuming for a surgeon to remove any resulting bone fragments from the body cavity.

Clamps that surgeons currently use to resect a proximal portion of the tibia include the Backhaus towel clamp, the Hoff towel clamp, and common haemostatic forceps. None of these clamps is designed to engage the proximal tibia piece that is resected during knee replacement surgery. These clamps, known in the prior art, may bend under excess force, require continued re-clamping to secure a good grip on the proximal portion of the tibia, deny the surgeon a high level of control, lack an adequate clamping range to engage the proximal portion of the tibia, or be generally too large or cumbersome to be practical for use in total knee replacement surgeries.

For example, the Backhaus towel clamp was designed to secure disposable towels or drapes to a patient during surgery. When the instrument is pulled horizontally in preparation for removal of the proximal tibia, the instrument's jaws can tear through the tibial bone. If the bone is cut by the clamp, the surgeon must re-clamp the instrument and remove any resulting bone particles from the body cavity. Another problem arises with the Backhaus clamp's effective clamping range, which is only about 1.0 to 5.0 mm, much smaller than the proximal portion of the tibia. Additionally, the Backhaus clamp is not designed to withstand the forces necessary to resect bone, and the instrument often deforms under the forces used to resect a portion of the tibia.

On the other hand, the Hoff towel clamp does not readily puncture bone, but it is not large enough to engage both sides of the tibia to allow for easy resection. The Hoff towel clamp does not have an adequate clamping range by which it can securely grip an object. Its locking mechanism has a very limited range of use, and when significant force is applied, the instrument can become damaged or bent. Furthermore, the Hoff clamp is difficult to use in knee surgery because its upper and lower jaws are equal in size, and each is difficult to insert beneath the tibia bone.

Haemostatic forceps are sometimes used to grab the cut portion of the proximal tibia, but such forceps were really designed to clamp off blood vessels and to stop bleeding. Haemostatic forceps have greater clamping surface area than the Backhaus or the Hoff clamps, and these forceps have straight jaws that function like scissors. But the straight jaws of haemostatic forceps are not capable of making full contact with the tibial bone segment because to maximize contact with the object, the clamp works best with objects of only about 1.0 to 3.0 mm of thickness.

Bone holding forceps can also be used to remove the tibia head, but they have no finger holes to help control the instrument in the incision. Bone forceps generally will not break a tibial portion, but they do not have the ability to grasp both tibia plateaus simultaneously, and they lack the refinement to clear the tibial spines, which may result in breaking the spines and producing bone fragments within the patient's body cavity.

Recent innovations in the art have led to new procedures such as mini total knee replacement. In a mini total knee replacement, arthroplastic surgery is performed through a significantly smaller incision than that through which a traditional knee replacement is performed. Mini total knee replacement spares cutting of the quadricep tendon, thereby reducing patient recovery time, but requiring that a surgeon use different tools than he would during a traditional knee replacement procedure in order to confine his work to a smaller space. The available surgical operating space in a mini total knee replacement using the quad-sparing technique is typically one-third to one-half the size of the space that would be available to a surgeon during a traditional total knee replacement surgery. As such, refined tools are needed to complete specific tasks in knee arthroplasty.

### BRIEF SUMMARY OF THE INVENTION

The surgical bone clamp has a large surface area to engage, for example, the proximal portion of the tibia that is removed during knee surgery. The bone clamp is lightweight, is easily maneuverable and is designed to withstand the forces required to resect a portion of the proximal tibia in knee surgery. The bone clamp is of simple construction and provides a device that is economically feasible, long lasting and relatively trouble free in operation.

The bone clamp includes a handle portion, a ratchet locking portion, and a clamping portion. The handle portion includes first and second handle arms, pivotally connected to one another between first and second ends of each. Finger loops are provided at the first end of each handle arm for receiving a thumb or at least one opposing finger, such as a forefinger. Cooperating components of the ratcheting mechanism are carried on each finger loop proximate the first end of each, permitting a stepwise reduction in distance between the finger loops and between the second ends of the handle arms through the pivotal connection. The clamping portion comprises two serrated jaws, a split top jaw and a full bottom jaw, that engage the tibial bone piece and that facilitate resection of the proximal tibial head in total knee arthroplasty.

### BRIEF DESCRIPTION OF THE DRAWINGS

FIG. 1 illustrates a perspective view of the surgical bone clamp.

FIG. 2 illustrates a perspective view of the clamping portion.

FIG. 3 illustrates a perspective view of the top clamping jaw.

FIG. 4 illustrates a perspective view of the bottom clamping jaw.

FIG. 5 illustrates a side view of the clamping portion.

FIG. 6 illustrates a perspective view of the handle portions.

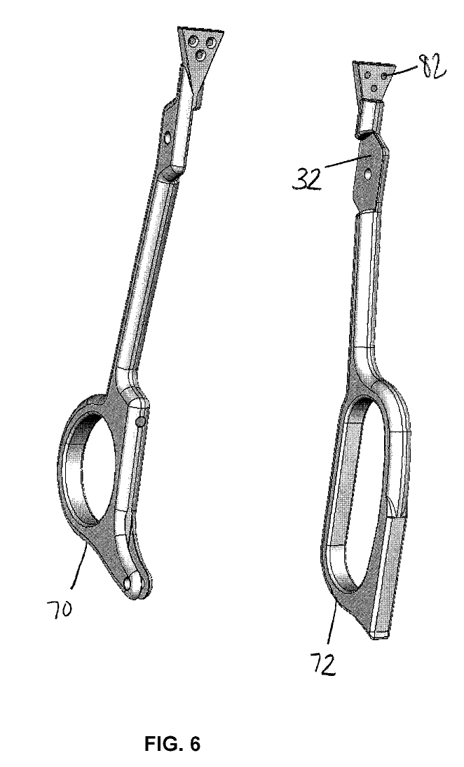

FIG. 7 illustrates a perspective view of the top clamping jaw.

### DETAILED DESCRIPTION OF THE EXEMPLARY EMBODIMENTS

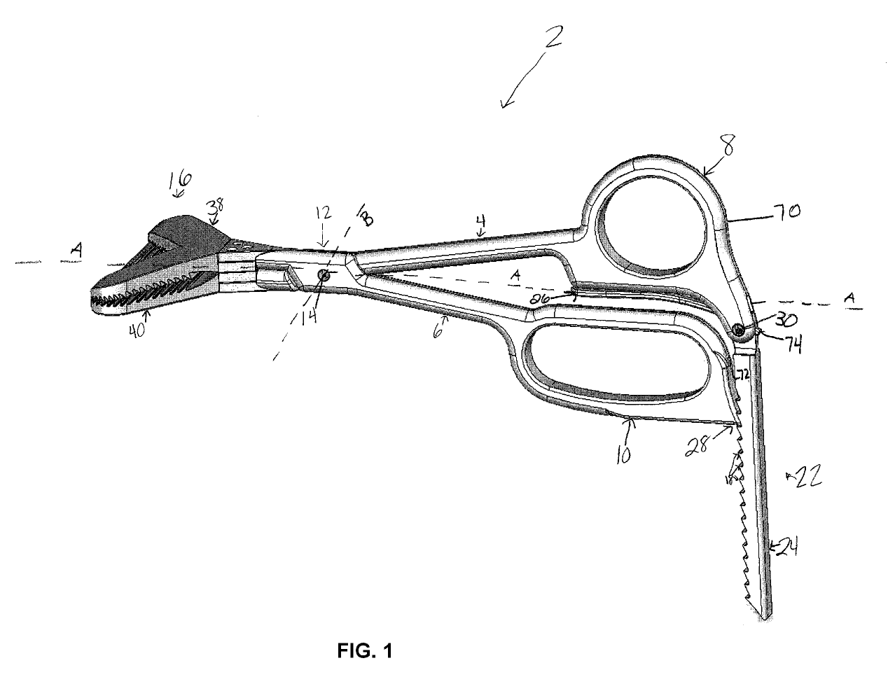

FIG. 1 illustrates generally a bone clamp 2 in accordance with the invention. The components of the bone clamp 2 are constructed of a suitable material that will allow for sterilization, will resist corrosion, and is of the type used for instruments of similar application, such as, for example, stainless steel.

A pair of handle arms 4 and 6 is hingedly and pivotally connected at a pivot joint 12 by a pivot pin 14. Handle arm 4 will be referred to as the first handle arm and handle arm 6 will be referred to as the second handle arm. The handle arms 4 and 6 are used to hold the device as well as manipulate the bone engaging means 16 within the surgical space. The pivot pin 14 forms an axis of rotation of the relative movement between the handle arms 4 and 6 so that the arms can pivot in a normal scissors or forceps fashion.

Finger loops 8 and 10 are provided at respective ends of handle arms 4 and 6 for transmission of a hand motion to the handle arms 4 and 6 and transmission, then, to the clamping jaws 38 and 40. Finger loop 8 will be referred to as the first finger loop, and finger loop 10 will be referred to as the second finger loop. The first finger loop 8 is designed to engage a thumb in the preferred embodiment and is of generally circular shape, while the second finger loop 10 is oval shaped and simultaneously engages at least one opposite finger, such as a forefinger. The user can grasp the handle portion and finger loops 8 and 10 as desired. This particular design will provide the user with a comfortable grip as well as an advantageous means for manipulating and handling the bone clamp 2 with only one hand.

In the preferred embodiment, the finger loops 8 and 10 are closed loops, allowing the user to effect a closure of the bone clamp 2 wherein finger loops 8 and 10 are brought together and handle arms 4 and 6 move to a position in which each approaches a common longitudinal handle axis A. Longitudinal handle axis A is substantially transverse to pivot pin axis B. The distal ends 70 and 72 of the finger loops 8 and 10 are approximately collinear when they are approximately parallel. Similarly, opening of the bone clamp 2 is affected by movement increasing the distance between finger loops 8 and 10. The finger loops 8 and 10 are located at the opposite end of the clamp from the bone engaging means 16.

The ratchet arm 22 extending from the first finger loop 8 comprises a series of teeth 18, each tooth having a face relatively perpendicular to the longitudinal axis of the ratchet. The perpendicular faces are on a side of the ratchet arm 22 facing the finger loops 8 and 10. The other side 24 of the ratchet arm 22 has no teeth. The top 74 of the ratchet arm 22 is machined to make contact with a piece of spring steel 26 that applies a force against the top of the ratchet arm 22 and forces it against the pawl 28, allowing the ratchet arm 22 to lock into place. This configuration allows for stepwise closure of the handle arms 4 and 6 as the pawl 28 slips over the sloped surface of each of the teeth 18 on the ratchet arm 22, while the surface of each of the teeth engages the pawl 28 to prevent opening of the handle arms 4 and 6. Successive ratchet teeth 18 are separated by a fixed linear distance.

The pawl 28 extends from the second finger loop 10. The ratchet arm 22 is pivotally connected to the first finger loop 8 with a pivot bolt 30 and is free to swing on the pivot bolt 30. Alternatively, the ratchet arm could be integral to the first handle arm 4. Opening of the clamping jaws 38 and 40 and separation of the handle arms 4 and 6 is achieved by disengaging the ratchet arm 22 from the pawl 28. In the preferred embodiment, the ratchet arm 22 may be locked or disengaged by the user with only one hand and even using a single finger of the same hand that is applying the clamping force.

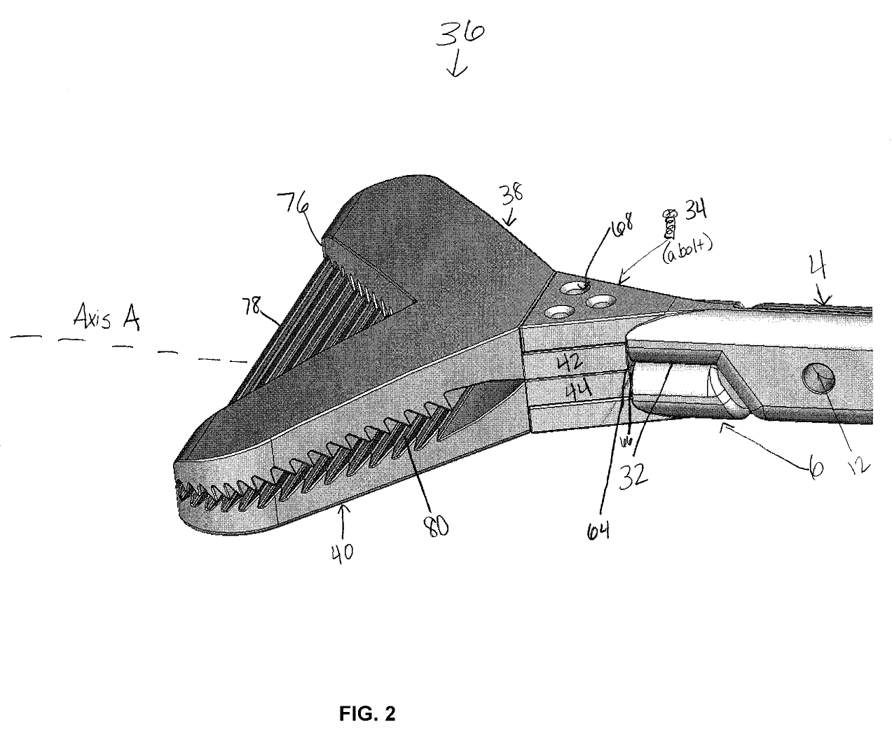

FIG. 2 illustrates the clamping portion 36, which comprises, primarily, a top jaw 38 and a bottom jaw 40. As shown in FIG. 2, the handle arms 4 and 6 extend past the pivot joint 12 that connects the handle arms to engage one another along a planar interface 32, perpendicular to the bolts 34 which hold the top jaw 38 and the bottom jaw 40 in place. This planar interface 32 provides stability and lessens the likelihood that bowing of the handle portion will occur when the clamp is exposed to large amounts of force.

Each of the handle arms 4 and 6 provides through holes 68 that allow for attachment of the top and bottom jaws 38 and 40. The jaws 38 and 40 are wider than the handle arms 4 and 6. The top jaw 38 has corresponding holes 50 that align with the holes 68 of the first handle arm 4. The bottom jaw 40 has corresponding holes 52 that align with the holes 82 of the second handle arm 6. In the preferred embodiment, the top jaw 38 and the bottom jaw 40 have threads cut into their planar portions 42 and 44 to allow bolts 34 to pass through the planar portion of the handles 32 and thread into the jaws 38 and 40, connecting the jaws 38 and 40 to the handle arms 4 and 6. In one embodiment, three holes 68 are provided, and the jaws are secured to the handle with three bolts 34 in order to prevent torque on the jaws 38 and 40 when force is applied. Other methods of attachment could be used to connect the jaws 38 and 40 to the handle arms 4 and 6, including methods that allow for some rotation of the jaws 38 and 40 along the longitudinal axis A of the bone clamp.

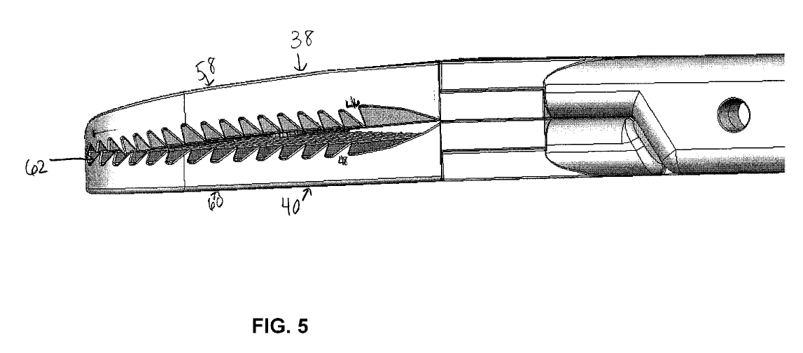

As shown in FIG. 5, the clamping jaws 38 and 40 include a roughened surface 80 so as to enable them to secure a bone portion within the clamp 2 between the jaws 38 and 40. In the preferred embodiment, the surface of each jaw 38 and 40 is serrated, having teeth 62 to grasp a bone piece and so as to be highly resistive to movement of the bone toward the terminal ends 76 and 78 of the jaws 38 and 40. Each jaw 38 and 40 has a smooth outer side 58 and 60 and a serrated side 46 and 48. The teeth 62 provide a secure grip on the bone, slightly penetrating the bone surface when clamped on the bone piece. The teeth 62 may be back-angled approximately forty-five (45) degrees from the surface of the jaws 38 and 40 to further help prevent slippage. Teeth of varying sizes may be incorporated to better grip the bone piece, and the angle of the teeth 62 relative to the surface of the jaws 38 and 40 may measure up to ninety (90) degrees.

Both the top and bottom jaws are removable and replaceable and may be exchanged for jaws of different sizes to meet the needs of a particular patient or procedure. In one embodiment, each jaw measured 4.013 cm in length from the end of the connecting planar portion 64 and 66 to terminating end 76 and 78 and 6.35 cm wide across the terminating end 76 and 78. These dimensions maximize grasping surface area but keep the overall size of the clamp 2 small so as to be useful in surgeries such as mini total knee replacement. In one embodiment, the top jaw 78 reaches 75% of the way across the proximal portion of the tibia on an average person.

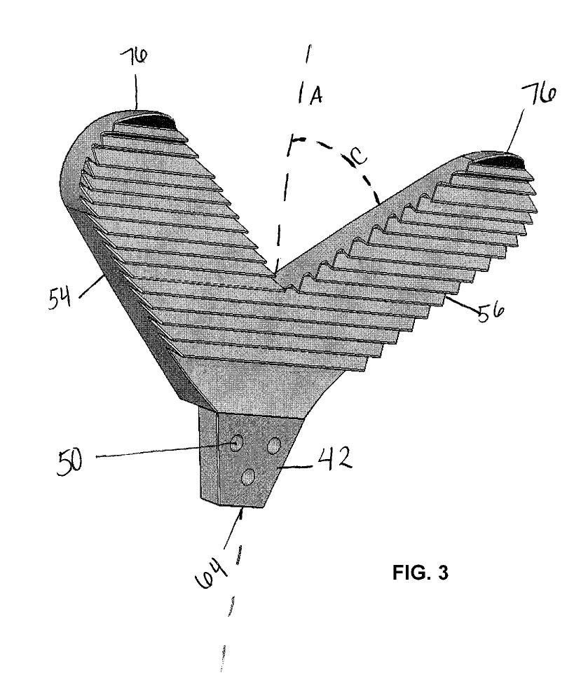

The top jaw 38, as seen in FIG. 3, is designed to accommodate a bone piece such as the proximal portion of the tibia. The top jaw 38 is split to create a Y-shape which allows the jaw to clear the central tibial spines and to clamp onto the two plateaus of the proximal tibia. The split in the top jaw 38 creates two jaw legs 54 and 56. In one embodiment, the angle between the legs of the top jaw 54 and 56 is 105.53° to accommodate the tibial spines. The angle between the legs of the top jaw 54 and 56 may vary according to patient size. The angle C between the longitudinal axis A and a jaw leg 54 does not exceed ninety (90) degrees.

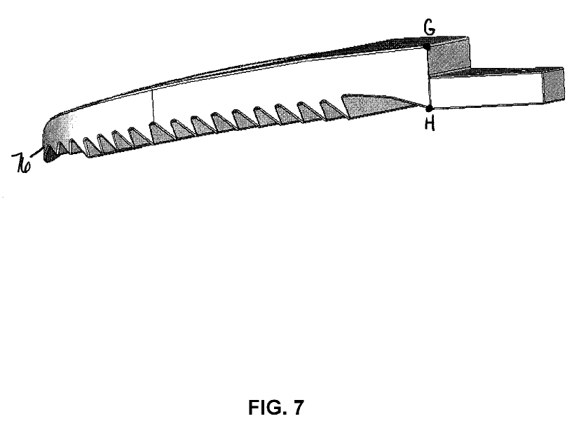

The top jaw 38 has large gripping surface area to allow a gripping force to be distributed over the entire area to prevent the jaw from breaking the bone portion. Because the instrument was designed to accommodate bones of varying thicknesses, the top jaw has a generally convex, arced shape to maximize instrument-to-bone gripping contact when the jaws are open. This arced shape is illustrated on FIG. 7 where the arc length from point G to the terminal end 76 of the top jaw is greater than the arc length from point H to the terminal end 76 of the top jaw, allowing the bone clamp 2 to best grip bone pieces up to 2.0 cm thick. This does not prevent the clamp from gripping bone pieces that are larger or smaller than average, but clamping surface contact with the bone piece is maximized when the bone piece is between 0.5 cm and 1.5 cm thick. The clamp is designed so that clamp surface area contact with the bone piece is maximized when the clamp is used on the average person.

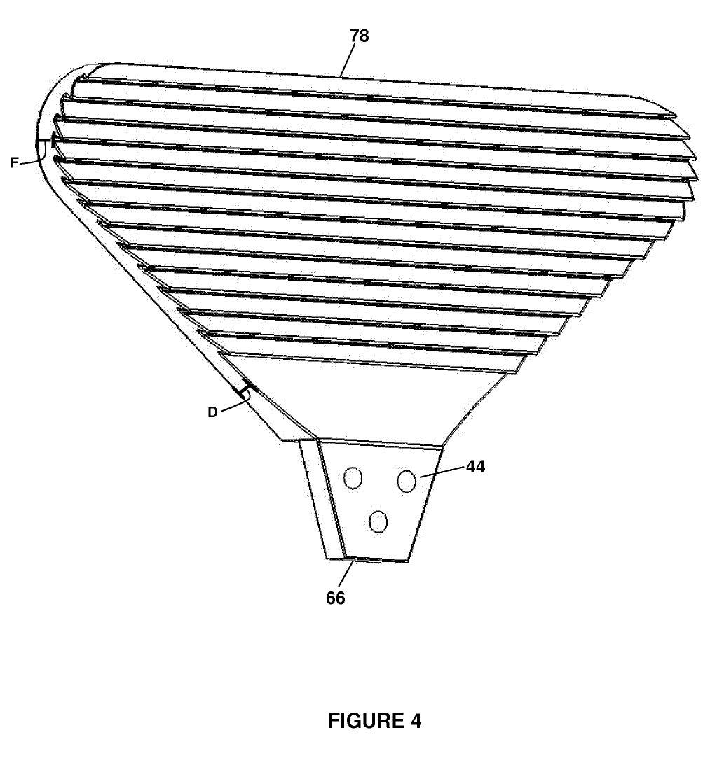

FIG. 4 illustrates the broad bottom jaw 40. The bottom jaw 40 functions as a broad surface against which the top jaw 38 can exert its gripping force. The bottom jaw 40 must be thick enough to have a significant force applied to it yet thin enough not to unintentionally dislodge the tibial head during surgery. In the preferred embodiment, the bottom jaw 40 is approximately 4.0 mm thick, having a tapered tip that allows it to easily slide under the cut portion of the proximal tibia bone. The tapered tip is illustrated in FIG. 4 where distance D is greater than distance F. The bottom jaw 40 has the same length and width dimensions as the top jaw 38, except that it is roughly triangular in shape and there is no split in the bottom jaw 40.

In the preferred embodiment, the bone clamp 2 is operated by inserting a thumb into the first finger loop 8 and at least one opposing finger into the second finger loop 10 and closing the handle arms 4 and 6 by moving the handle arms together in response to applied pressure. The pivot pin 14 allows for the movement of the handle arms 4 and 6 to be transferred into the clamping jaws 38 and 40. Meanwhile, the ratchet arm 22 and pawl 28 allow for stepwise movement in a closing direction. The distance between the pivot pin 14 and the terminal ends 76 and 78 of each of the top and bottom jaws 38 and 40 is substantially shorter than the distance between the pivot pin and the terminal ends 70 and 72 of each of the finger loops 8 and 10 in order to obtain an increase in mechanical advantage, facilitating the application of increased pressure on the bone piece as the clamp is closed.

Many additional configurations may be adapted to suit particular applications. The bone clamp has been described with respect to the resection of a proximal portion of the tibia because of its particular advantages in such application. However, it will be understood that the present invention may also be used with equal utility for the resection of other bone pieces or fragments. In such cases, the dimensions of the bone clamp would be increased or changed appropriately.

Having described preferred embodiments of the invention with reference to the accompanying drawings, it is to be understood that the invention is not limited to those precise embodiments, and that various changes and modifications may be effected therein by one skilled in the art without departing from the scope or spirit of the invention as defined in the appended claims. A number of modifications may be made to the present invention without departing from the inventive concept therein. Various modifications to the preferred embodiment may be made within the scope of the present invention.

What is claimed is:

1. A surgical bone clamp, comprising:
a first handle arm;
a second handle arm;
a top clamping jaw connected to said first handle arm;
a bottom clamping jaw of a different shape than said top jaw that is connected to said second handle arm;
and a ratchet attached to one of said handle arms for providing stepwise engagement of said first and second handle arms from a position in which said jaws are widely separated to one in which said handle arms are brought closer together, while preventing movement in an opposite direction.

2. The bone clamp as recited in claim 1 wherein each handle arm has a finger engaging portion.

3. The bone clamp as recited in claim 1 wherein said top jaw is removable from said first handle arm.

4. The bone clamp as recited in claim 1 wherein said bottom jaw is removable from said second handle arm.

5. The bone clamp as recited in claim 1 wherein said top jaw is arced.

6. The bone clamp as recited in claim 1 wherein said top jaw has two jaw legs.

7. The bone clamp as recited in claim 1 wherein said bottom jaw has a tapered end.

8. The bone clamp as recited in claim 1 wherein the top jaw has a surface shaped to engage the plateaus of the proximal tibia.

9. The bone clamp as recited in claim 1 wherein the top jaw has a rough surface.

10. The bone clamp as recited in claim 1 wherein the bottom jaw has a rough surface.

11. A surgical bone clamp, comprising:
a first handle arm;
a closed finger loop at an end of said first handle arm to engage the thumb;
a second handle arm;
a second closed finger loop at an end of said second handle arm to engage at least one finger;
a Y-shaped top jaw connected to said first handle arm; and
a tapered bottom jaw connected to said second handle arm;

12. The bone clamp as recited in claim 11 wherein said top jaw has a rough surface.

13. The bone clamp as recited in claim 11 wherein said bottom jaw has a rough surface.

14. The bone clamp as recited in claim 11 wherein said handle arms are pivotally connected.

15. The bone clamp as recited in claim 11 wherein the top jaw and the handle arms each have a width and the top jaw is wider than the handle arms.

16. The bone clamp as recited in claim 11 wherein the bottom jaw is tapered.

17. The bone clamp as recited in claim 11 wherein a ratchet portion is attached to one of the handle arms and a pawl is attached to the other of the handle arms, the pawl engageable with the ratchet for providing stepwise engagement of said first and second handle arms from a position in which said jaws are widely separated to one in which said handle arms are brought closer together, while preventing movement in an opposite direction.

18. A surgical bone clamp comprising:
a first handle arm;
a second handle arm pivotally connected to the first handle arm;
a top clamping member attached to one of said handle arms;
a bottom clamping member attached to the other of said handle arms;
and a top clamping member with at least two legs which straddle a longitudinal axis, each of the arms angled at less than ninety (90) degrees from the longitudinal axis.

19. The bone clamp as recited in claim 18 wherein the bottom jaw is of generally triangular shape.

20. The bone clamp as recited in claim 18 wherein the top jaw has a plurality of teeth.

21. The bone clamp as recited in claim 18 wherein the bottom jaw has a plurality of teeth.

22. The bone clamp as recited in claim 18 wherein the top jaw is replaceable with a top jaw of a different size.

23. The bone clamp as recited in claim 18 wherein the bottom jaw is replaceable with another bottom jaw of a different size.

24. The bone clamp as recited in clamp 18 wherein the top jaw has an arced portion.
